(19) United States
(12) Patent Application Publication
(10) Pub. No.: US 2008/0312495 A1
(43) Pub. Date: Dec. 18, 2008

(54) Method of Performing Transgastric Abdominal Surgery

(76) Inventors: Dennis L. McWilliams, Austin, TX (US); Brett E. Naglreiter, Austin, TX (US)

Correspondence Address: GORDON & JACOBSON, P.C., 60 LONG RIDGE ROAD, SUITE 407, STAMFORD, CT, 06902, US

(21) Appl. No.: 11/761,428

(22) Filed: Jun. 12, 2007

(51) Int. Cl. A61B 1/273

(52) U.S. Cl. 600/104

(57) ABSTRACT

A minimally invasive method of performing surgical procedures in the peritoneum is provided. The method includes providing a non-lumened flexible visualization scope, a flexible gastric pressurization tube, and at least one additional flexible manipulating instrument, wherein the scope, tube and the instruments are all discrete from each other and all smaller than 2 mm in diameter. The tools are extended into the esophagus and transgastrically through holes defined by incisions or instrument piercing in the stomach wall into the peritoneum, where they are used to visualize, insufflate and perform a peritoneal surgical procedure. Upon removal of the instruments, the holes are substantially self-sealing, and do not require surgical closure. Optionally, a biocompatible glue can be dispensed at the holes to facilitate closure.

### BACKGROUND OF THE INVENTION

1. Field of the Invention

This invention relates broadly to surgery. More particularly, this invention relates to methods of performing minimally invasive surgical procedures.

2. State of the Art

Throughout most of the history of surgery, general surgical procedures, i.e., surgical procedures performed within the abdomen, were performed open. That is, the abdominal cavity was surgically opened to expose the internal organs and provide direct access to the surgeons performing the surgical procedure. However, the trauma to the patient from the procedure can present significant issues for the patient, including extended recovery time. Moreover, there is the danger of complication as prolonged exposure of surgical wounds to the open environment heightens the chance of infections even in presumably sterile settings.

In the last few decades minimally invasive surgery has become possible and more and more important. A minimally invasive surgery is one in which the instruments enter the body through the skin or through a body cavity or anatomical opening with the smallest damage possible to these structures. Other than through the instrument passages, the body is not opened to the environment.

Minimally invasive surgery results in less operative trauma for the patient. It is also less expensive, reduces hospitalization time, causes less pain and scarring, and reduces the incidence of complications related to the surgical trauma, speeding the recovery. However, even in minimally invasive procedures, holes are made in the body that are of such significant dimension that they need to be closed after instrument removal.

For example, in a peritoneal laparoscopic procedure from outside the body, multiple holes will initially be made in the abdominal wall. These holes provide access for a visualization scope, a pressurization pathway to insufflate the abdomen, and two or manipulating instruments (e.g., graspers, forceps, scalpels, staplers, suturing devices, irrigators, cauterization devices, etc.) to be inserted into the abdomen. Given the sizes of the holes, the holes will need to be sutured or stapled closed at the conclusion of the procedure.

U.S. Pat. No. 5,458,131 to Wilk teaches an intra-abdominal method for performing peritoneal procedures. In such procedures, a flexible endoscope is extended through the esophagus and passed through an incised hole in the stomach wall into the peritoneum. Other flexible and steerable manipulating instruments are similarly passed through the lumen of the endoscope and other incised holes in the stomach wall or through an incised perforation in another natural body cavity. The endoscope includes a lumen that may be used for insufflation of the peritoneum or for passage of additional instruments. Such endoscopes are typically on the order of 9 mm to 15 mm, with lumen diameters of 2 mm to 5 mm. The instruments used therethrough are typically sized for close fit through the lumen of the endoscope. Upon completion of the surgery, after the instruments are withdrawn, the incised holes in each of the body cavities are surgically closed. U.S. Pat. No. 5,458,131 to Wilk teaches closure of the holes via ligation using O-rings.

The advantages of such minimally invasive surgery includes minimizing the trauma of access to internal organs and decreasing the time of the surgical procedure. By avoiding a long incision through the muscular abdominal wall, many post-operative problems are eliminated. Furthermore, the patient is provided with reduced anesthetic as the procedure is shortened and the trauma is decreased. The need for strong post-operative pain medications is drastically reduced so that the drowsiness, fatigue and unsteadiness they cause are virtually eliminated, and it is possible to return to normal activities in a fraction of the time necessary after regular surgery.

It is anticipated that further reducing trauma to the body during surgical procedures and reducing the time of a surgical procedure will provide better surgical outcomes and decreased recovery times.

### SUMMARY OF THE INVENTION

It is therefore an object of the invention to provide minimally invasive methods of performing surgical procedures in the peritoneum which are less invasive than currently performed minimally invasive methods.

It is another object of the invention to provide a minimally invasive method of performing surgical procedures in the peritoneum that reduces the extent of the internal incisions, particularly at the gastric wall, and thus reduces trauma to internal organs.

It is a further object of the invention to provide a minimally invasive method of performing surgical procedures in the peritoneum that does not necessitate surgical repair of holes upon withdrawal of the instruments.

In accord with these objects, which will be discussed in detail below, a minimally invasive method of performing surgical procedures in the peritoneum is provided. The method includes providing a non-lumened flexible visualization scope, a flexible gastric insufflation tube, and at least one additional flexible manipulating instrument, wherein the scope, tube and the instruments (collectively, ‘the surgical tools’) are all discrete from each other. The surgical tools do not exceed 2 mm in diameter, and are all preferably steerable or otherwise guidable without being extended through an endoscope. The tools are extended into the esophagus and transgastrically through incisions or piercings in the stomach wall into the peritoneum, where they are used to visualize, insufflate and perform a peritoneal surgical or diagnostic procedure. By way of example, such procedure may include cauterizing a site of abdominal bleeding, ablation of tumors, or local delivery of therapeutic agents. At the conclusion of the procedure, the tools are withdrawn. In view of the small dimension of each of the tools (significantly smaller than the incision required for the flexible lumened endoscope passed through the stomach wall in prior art transgastric peritoneal procedures), the holes are substantially self-sealing, and do not require surgical closure, as the holes will close on their own. Optionally, a biocompatible glue can be dispensed at the holes to facilitate closure. Such glue can be released from a discrete instrument or from portion of one or more of the tools upon removal of the tools from their respective holes.

Additional objects and advantages of the invention will become apparent to those skilled in the art upon reference to the detailed description taken in conjunction with the provided figures.

### BRIEF DESCRIPTION OF THE DRAWINGS

FIG. 1 through 5 schematically illustrate a first embodiment of a method of a transgastric peritoneal surgical procedure.

FIG. 6 is a distal end view of an exemplar tissue manipulating instrument having glue releasing capability.

FIG. 7 are broken side elevations of the distal ends of embodiments of instruments in an instrument set for carrying out a second embodiment of the invention.

FIG. 8 is a schematic illustration of instruments and methods for carrying out a third embodiment of the invention.

FIG. 9 is a schematic illustration of instruments and methods for carrying out a fourth embodiment of the invention.

FIG. 10 is a schematic illustration of instruments and methods for carrying out a fifth embodiment of the invention.

FIGS. 11 through 15 are schematic illustrations of instruments and methods for carrying out a sixth embodiment of the invention.

### DETAILED DESCRIPTION OF THE PREFERRED EMBODIMENTS

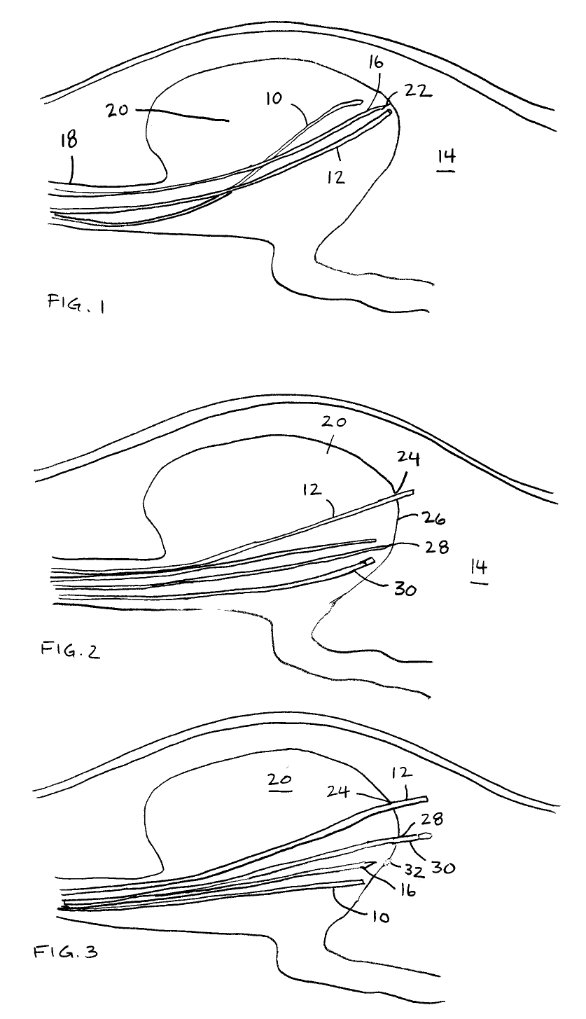

Turning now to FIGS. 1 through 5, a first embodiment of performing an transgastric peritoneal surgical procedure on a mammal and preferably a human according to the invention is shown. Referring to FIG. 1, in carrying out the method, a flexible steerable visualization scope 10, a flexible steerable insufflation tube 12 for insufflating the peritoneal space 14 and a flexible steerable endoknife (needle knife) 16 are inserted through the esophagus 18 and into the stomach 20. Exemplar instruments having suitable dimensions for carrying out the invention include the following: the visualization scope 10 can be a 1.8 mm CCD camera available from Medigus Ltd. of Israel, and the insufflation tube 12 can be a Cragg-McNamara 4 Fr or 5 Fr Valved Infusion Catheter available from ev3, Inc. (Neurovascular Division) of Irvine, Calif. While these commercially available devices are not steerable, modified versions of these devices may incorporate the steering technology used in commercially available endoscopes. Endoscope steering technology includes the use of pull wires to cause the distal end of the endoscope to bend in a desired direction. The endoknife may be a Needle Knife KD-10Q-1.A, available from Olympus America Inc, Melville, N.Y., having a diameter suitable for a 2.0 mm diameter endoscope working channel. The scope 10, tube 12 and the endoknife 16 all include elongate tubular bodies, and preferably the portions of such devices that are insertable into the intragastric cavity do not exceed 2 mm in diameter. More particularly, the distal ends of the scope 10, tube 12 and the endoknife 16 are all preferably 2 mm or smaller in diameter. These instruments have a length in the range of about 100 cm to about 300 cm with a preferred range of about 150 cm to 250 cm. The length of the instrument that extends across the gastric wall and into the peritoneal space is in the range of 25 cm to 100 cm. Referring to FIGS. 2 and 3, under visualization of the scope 10, the cutting tip 22 of the endoknife 16 is used to incise a first small hole 24 in the gastrointestinal wall 26 (preferably in the wall of the stomach 20). The incision 24 preferably does not exceed 2 mm in diameter. The insufflation tube 12 is maneuvered through the first hole 24 and into the peritoneal space 14 in the abdomen. The endoknife 16 is then moved to a second location and used to incise a second small hole at 28 (not exceeding 2 mm in diameter) for passing a manipulating instrument 30 (e.g., grasper, forceps, scalpel, cauterization device) into the peritoneal space 14. The process may be repeated for any additional instrument that may be required during the procedure. At any time during the procedure, the endoknife 16 is utilized to incise a hole 32 to insert the scope 10 into the peritoneal space 14. In addition, the endoknife 16 may incise a hole 34 (FIG. 5) for itself and then be passed through into the peritoneal space 14 for use in carrying out a procedure within the peritoneal space. Preferably none of the holes created in the gastrointestinal wall are larger than 2 mm in diameter.

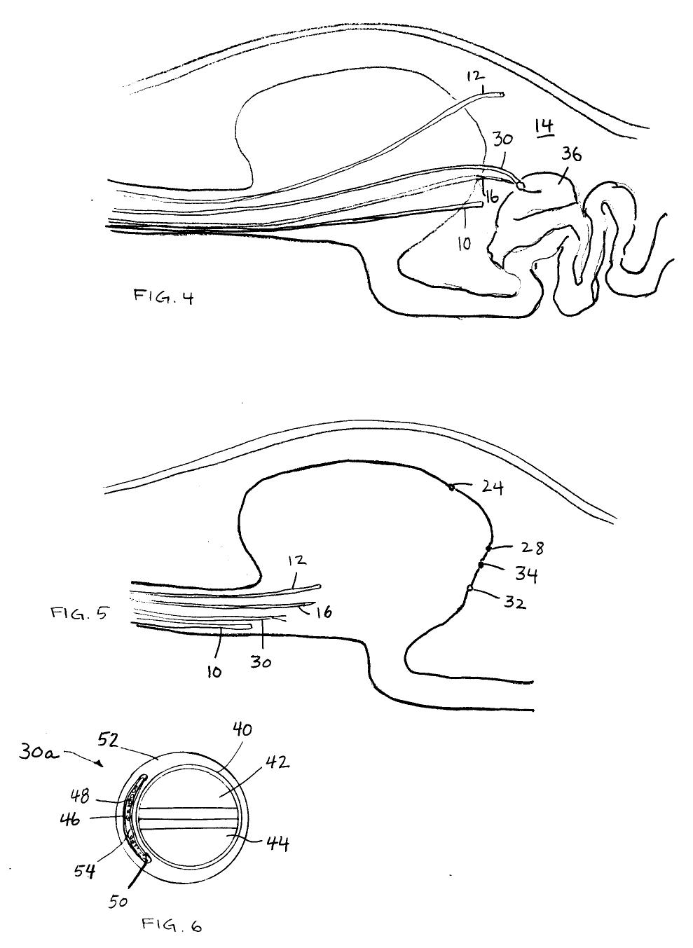

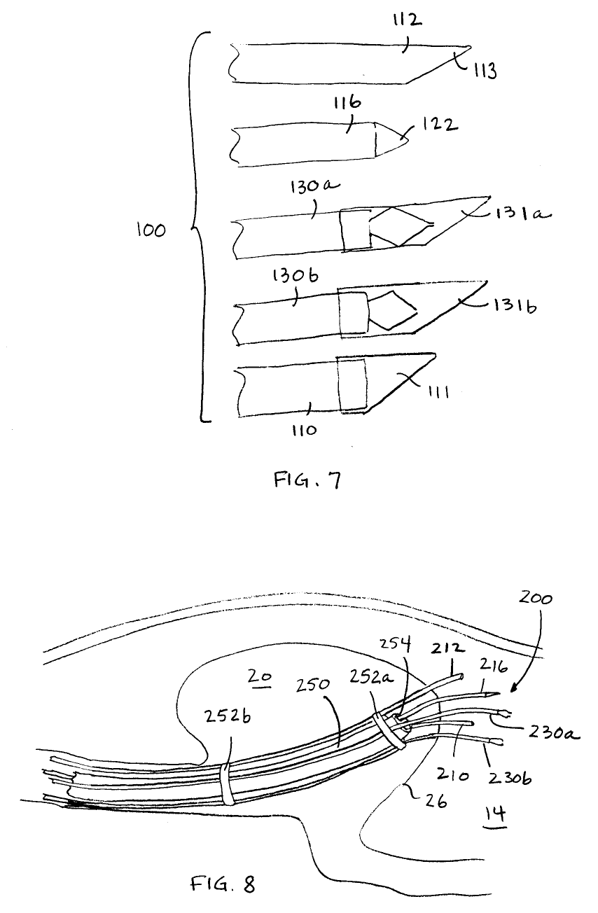

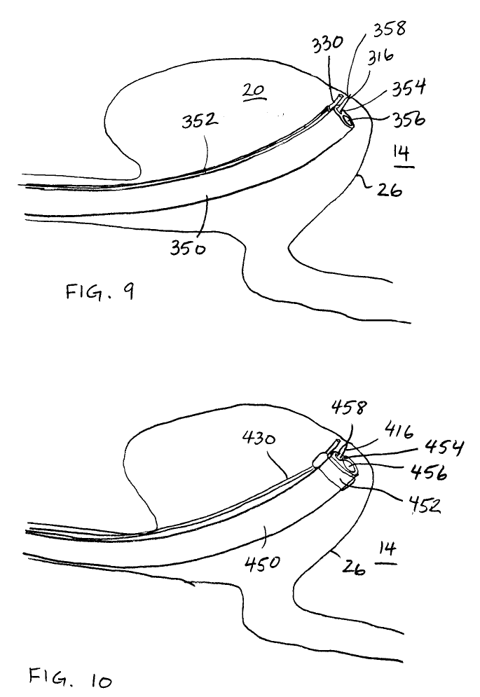

Referring to FIG. 4, after the insufflation tube 12 is within the peritoneal space 14, insufflation fluid is passed through the insufflation tube to expand the peritoneal space. Then, under observation of the scope 10, the manipulating instruments 16, 30 are actuated to operate on tissue 36 and perform a surgical procedure within the peritoneal space. Such procedures include obtaining a tissue sample, removal of a tumor or local delivery of a therapeutic agent. Referring to FIG. 5, once the procedure is complete, the instruments 10, 12, 16, 30 are removed from the peritoneal space, and withdrawn through the stomach 20 and esophagus 18. In view of the small diameter of each of the instruments that is passed through the gastrointestinal wall 26, the holes 24, 28, 32, 34 will self-seal quickly on their own.

If closure assistance is required or optionally desired for the holes, given the small diameter of the holes, such assistance can be provided via an adhesive (as opposed to mechanical devices that must penetrate pierce or tissue on opposite sides of the hole). In accord with one embodiment of the invention, a dedicated instrument or one or more of the previously identified instrument types is provided with a channel or small diameter lumen from which a tissue adhesive can be dispensed. It is appreciated that hole closure via an adhesive and without tissue penetration is significantly less time consuming and the system for dispensing an adhesive is significantly less complex than that required to manipulate tissue and to dispense staples, clips, ligating bands, etc.

For example, turning to FIG. 6, a distal end of a forceps device 30a is shown with a first lumen 40 for the forceps jaws 42, 44 and a second lumen 46 through which adhesive 48 can be dispensed. The adhesive 48 can be contained in a reservoir 50 near the distal end 52 of the device 30 and pushed out with an advancing shovel 54 attached to a wire longitudinally actuated from the proximal end of the instrument. A frangible membrane may be provided over the distal end of the second lumen reservoir 50 and automatically removed under pressure as the shovel 54 is distally advanced to release the adhesive 48.

Turning now to FIG. 7, a set 100 of instruments is shown for carrying out another embodiment of a method according to the invention. Each instrument in the set 100 is provided with a tissue piercing end that can pierce the tissue of the gastrointestinal wall without prior incision with an endoknife. Visual scope 110 includes a chisel-cut tissue piercing hood 111. The hood 111 is preferably retractable relative to the distal end of the scope to provided unencumbered visualization of the peritoneum after passage through the gastrointestinal wall. Insufflation tube 112 includes an angle-cut tissue piercing end 113. Endoknife 116 has a sharp cutting end 122. Graspers 130a and biopsy forceps 130b are each provided with retractable hoods, 131a, 131b, respectively, similar to hood 111, but which can also operate as jaw release and jaw closure means. Thus, each of the instruments 110, 112, 131a, 131b can be forced directly through the gastrointestinal wall and into the peritoneal space without previously defining a hole for passage therethrough with the endoknife 116. It is appreciated that other tissue piercing means can be provided to the individual instruments in the set.

In view of the above, it is a goal of the invention to operate intragastrically and within the peritoneal space without necessitating incision of one or more large holes for passage of endoscopes or other relatively large instruments that have been previously required for such surgery. The prior use of endoscope in such surgery includes scopes substantially larger than 2 mm in diameter and defining one or more lumen for the passage of instruments. Nevertheless, as described hereinafter, it is appreciated that the surgery of the invention may be facilitated with the use of an endoscope larger than 2 mm in diameter passed through the esophagus and into the stomach, but not through the gastrointestinal wall.

Referring now to FIG. 8, another embodiment of a method according to the invention is shown. The instruments 210, 212, 216, 230a, 230b (collectively 200) for passage into the peritoneal space 14 are retained about the periphery in a predetermined radial proximity relative to substantially larger endoscope 250. Such relationship is maintained using, for example, a spacer 252a. The spacer 252a preferably maintains a relative distance between the instruments 200 that are to be passed into the peritoneal space 14. Moreover, the spacer 252a (and several spacers 252b may be provided along the length of the larger endoscope 250) increases the effective longitudinal stiffness of the instruments 200 as they are forced through the gastrointestinal wall 26 and provides increased stability as they are actuated. The instruments 200 may be moved longitudinally relative to the spacer(s) 252a, 252b to operate within the peritoneal space 14. In addition, the larger endoscope 250 provides visualization of the gastrointestinal space, e.g., stomach 20, even after a smaller endoscope 210 (2 mm or less in diameter) is passed into the peritoneal space. Optionally, the endoknife 216 can be passed through the lumen 254 of the endoscope 250, which provides stable guidance to the endoknife as the endoknife incises holes for the other instruments 200. Different instruments can also be extended through lumen 254. While endoscope 250 remains within the stomach cavity, or elsewhere in the intragastric cavity, a small scope not exceeding 2 mm in diameter (not shown) will extend transgastrically into the peritoneum 14 as previously described for visualization of the peritoneal procedure.

Turning now to FIG. 9, another embodiment of a method of the invention in which a standard gastrointestinal endoscope 350 is inserted into the stomach 20. The endoscope 350 includes a peripheral longitudinal channel 352 that at least partially retains an instrument 330 relative to the endoscope. The instrument can be moved longitudinally within the sheath 352 and the distal of the instrument can be inserted through the gastrointestinal wall 26 and into the peritoneal space 14 under observation of the optics 356 of the endoscope 350. The lumen 354 of endoscope 350 can be used for passage and guidance of an endoknife that incises a hole 358 for the instrument, or for other instruments. Additional instruments (not shown) can be inserted into the peritoneal space 14 coupled to other sheaths of the endoscope or in a manner discretely from the endoscope. While endoscope 350 remains within the stomach cavity, or elsewhere in the intragastric cavity, a small scope not exceeding 2 mm in diameter (not shown) will extend transgastrically into the peritoneum 14 as previously described for visualization of the peritoneal procedure.

Referring now to FIG. 10, another embodiment of a method of the invention in which a standard gastrointestinal endoscope is inserted into the stomach is provided. An instrument 430 is coupled to the distal end of the endoscope 450 with a cuff 452 that retains the distal end of the instrument 430 relative to the endoscope. The instrument 430 can be moved longitudinally within the cuff 452 and the distal of the instrument 430 can be inserted through the gastrointestinal wall 26 and into the peritoneal space 14 under observation of the optics 456 of the endoscope 450. The lumen 454 of endoscope 450 can be used for passage and guidance of an endoknife 416 that incises a hole 458 for the instrument 430, or for another instrument. Additional instruments (not shown) can be inserted into the peritoneal space 14 coupled to other cuffs attached to the endoscope or in a manner discretely from the endoscope. While endoscope 450 remains within the stomach cavity, or elsewhere in the intragastric cavity, a small scope not exceeding 2 mm in diameter (not shown) will extend transgastrically into the peritoneum 14 as previously described for visualization of the peritoneal procedure.

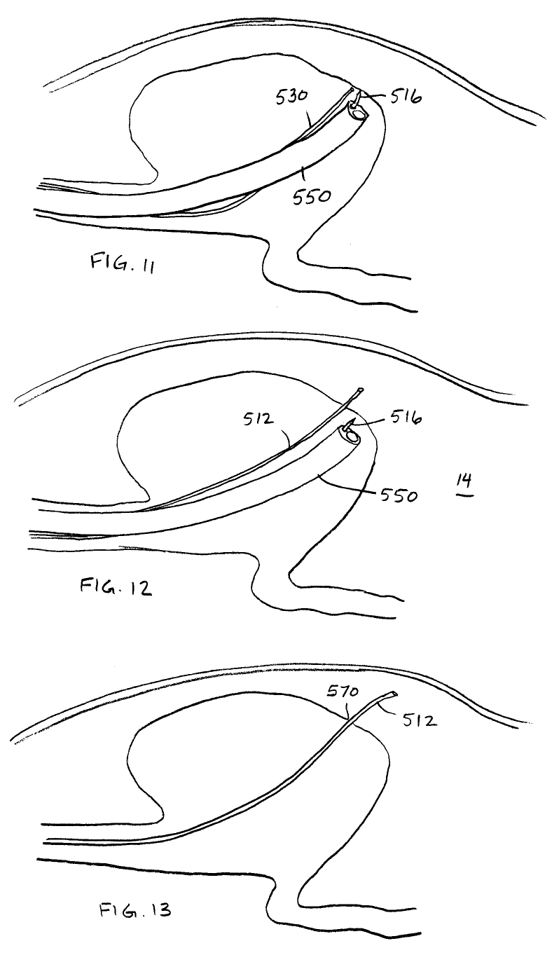

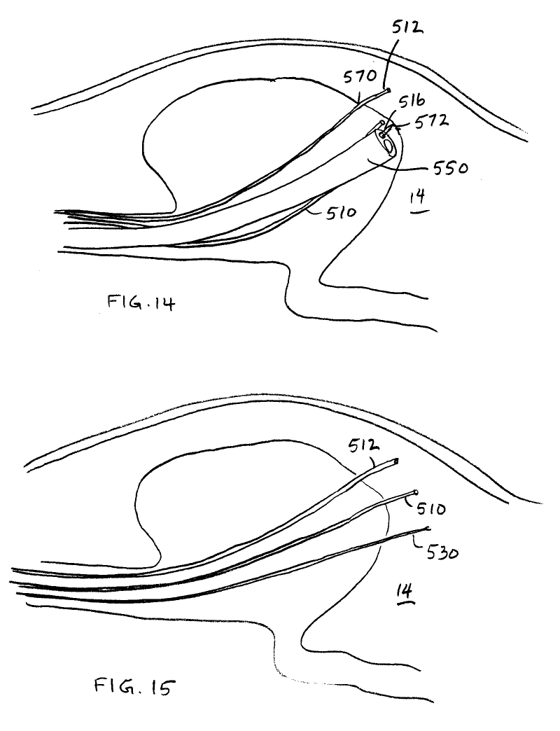

Turning now to FIGS. 11 through 15, another method according to the invention is shown. An endoscope 550 with a lumen 554 is extended through the esophagus and passed into the stomach. An endoknife 516 may be inserted through the lumen of the endoscope, with the endoscope 550 providing visualization and facilitating stabilization of the instrument 516. Instruments, e.g., scope 510, insufflation tube, and forceps 530 for operating or facilitating the operation within the peritoneal space 14 are also passed through the esophagus and into the stomach and through incisions 570, 572 made with the endoknife 516. Once all the necessary instruments for performing the peritoneal procedure are within the peritoneal space, the larger endoscope 550 can be removed from the gastric cavity and esophagus. Alternatively, the endoscope 550 can be left within the stomach to provide visualization of the gastric wall from within the gastric cavity. Once the peritoneal procedure is complete, the instruments are withdrawn from the patient's body.

There have been described and illustrated herein several embodiments of instruments for performing transgastric peritoneal surgery and methods of performing such surgical procedures. While particular embodiments of the invention have been described, it is not intended that the invention be limited thereto, as it is intended that the invention be as broad in scope as the art will allow and that the specification be read likewise. Thus, while a particular order to the insertion of various instruments has been disclosed, it will be appreciated that the instruments may be inserted in an order other than as described. In addition, while particular types of peritoneal procedures have been described, it will be understood that other procedures can be performed as well. Also, while particular tissue manipulating instruments have been described, it will be recognized that other tissue manipulating instruments can be inserted transgastrically into the peritoneal space for performing the procedures. It will therefore be appreciated by those skilled in the art that yet other modifications could be made to the provided invention without deviating from its spirit and scope as claimed.

What is claimed is:

1. A method of performing an transgastric peritoneal surgical procedure on a body, comprising:
a) inserting a flexible visualization scope into the esophagus, through a portion of the gastrointestinal wall and into the peritoneal space;
b) inserting a flexible insufflation tube into the esophagus, through a portion of the gastrointestinal wall and into the peritoneal space through which an insufflation fluid may be passed into the peritoneal space;
c) inserting a flexible first tissue manipulating instrument into the esophagus, through a portion of the gastrointestinal wall and into the peritoneal space,
wherein insertion of the scope, the tube and the first instrument does create any holes between the gastrointestinal wall and the peritoneal space larger than 2 mm in diameter,
d) inflating the peritoneal space with insufflation fluid;
e) actuating the first instrument to manipulate tissue in the peritoneal space under visualization of the scope; and
f) removing the first instrument, tube and scope from the body.

2. A method according to claim 1, further comprising:
using a flexible knife inserted into the body intragastrically through the esophagus to create holes for insertion of the scope, insufflation tube and first instrument into the peritoneal space.

3. A method according to claim 1, wherein:
each of the scope, tube and first instrument have a diameter not exceeding 2 mm.

4. A method according to claim 1, further comprising:
inserting at least one additional tissue manipulating instrument into the esophagus, through a portion of the gastrointestinal wall and into the peritoneal space; and
actuating said at least one additional instrument to manipulate tissue in the peritoneal space.

5. A method according to claim 1, further comprising:
introducing a second flexible visualization scope into the esophagus and into the stomach, but not into the peritoneal space.

6. A method according to claim 5, wherein:
said second scope includes a lumen for passage of an instrument.

7. A method according to claim 5, wherein:
said second scope include means for partially retaining a portion of at least one of said other scope, said insufflation tube, and said first instrument within a predetermined radial proximity.

8. A method according to claim 7, wherein:
said means includes one of a longitudinal slot, a distal cuff, and a distal ring with instrument spacing holes.

9. A method according to claim 1, further comprising:
releasing an adhesive glue at least one of said holes to facilitate closure of said hole.

10. A method according to claim 1, wherein:
said scope, said insufflation tube and said first tissue manipulating instrument are pierced through the gastrointestinal wall.

11. A kit for performing a minimally invasive surgical procedure transgastrically and within the peritoneal space, comprising:
a) a flexible visualization scope having a distal end with a diameter not exceeding 2 mm in diameter;
b) a flexible valved tubular member having a distal end with a diameter not exceeding 2 mm in diameter; and
c) a flexible cutting device having a distal end with a diameter not exceeding 2 mm in diameter.

12. A kit according to claim 11, further comprising:
at least one additional tissue manipulating instrument having a distal end with a diameter not exceeding 2 mm in diameter.

13. A kit according to claim 12, wherein:
at least one of said elements in said kit includes means for dispensing an adhesive to body tissue.

14. A kit according to claim 11, wherein:
at least one of said elements in said kit includes means for dispensing an adhesive to body tissue.

15. A kit according to claim 11, further comprising:
a instrument that dispenses a tissue adhesive.
